(19) United States
(12) Patent Application Publication
(10) Pub. No.: US 2008/0027365 A1
(43) Pub. Date: Jan. 31, 2008

(54) HEMOSTATIC DEVICE WITH OXIDIZED CELLULOSE PAD

(76) Inventor: Raymond J. Huey, Orange, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/754,592

(22) Filed: May 29, 2007

Related U.S. Application Data
(60) Provisional application No. 60/810,447, filed on Jun. 1, 2006.

(51) Int. Cl. A61F 13/02

(52) U.S. Cl. 602/44; 602/43; 602/45; 602/48; 602/53; 602/54; 602/56

(57) ABSTRACT

In one aspect, an apparatus for promoting hemostasis includes oxidized cellulose in the form of a compressible, shapeable mass that is formed into a sheet for placement on a bleed site. In a second aspect, a sleeve for promoting hemostasis includes a tubular shell having at least one open end and oxidized cellulose material attached to an inner surface of the tubular shell. The open end of the tubular shell is dimensioned to receive a limb such that donning the sleeve causes at least a portion of the oxidized cellulose material to be placed in contact with a bleeding wound. In a third aspect, a bandage includes a substrate and oxidized cellulose material attached to a surface of the substrate. The oxidized cellulose material is arranged to contact a bleeding wound when the bandage is applied thereto.

### CROSS-REFERENCE TO RELATED APPLICATION

This application claims the benefit of U.S. Provisional Patent Application No. 60/810,447, filed on Jun. 1, 2006, the contents of which are incorporated herein by reference in their entirety.

### TECHNICAL FIELD

The present invention relates generally to hemostatic devices and, more particularly, to devices that allow hemostatic materials incorporated therein to be brought into contact with wound sites.

### BACKGROUND OF THE INVENTION

Blood is a liquid tissue that includes red cells, white cells, corpuscles, and platelets dispersed in a liquid phase. The liquid phase is referred to as plasma and includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances humans (as well as animals) can be wounded, thereby resulting in bleeding. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stop the flow of blood in a timely manner.

In an effort to address the above-described problems, materials and devices have been developed for controlling excessive bleeding in situations where conventional aid is unavailable or less than optimally effective. Although these materials and devices have been shown to be somewhat successful, they are sometimes not effective enough for traumatic wounds and tend to be expensive. Furthermore, these materials are sometimes ineffective and can be difficult to apply as well as remove from a wound.

In situations in which traumatic wounds are experienced (such as automobile or motorcycle accidents) and where multiple wounds are inflicted on a victim over multiple portions of the victim's body, some wounds may be inflicted on both the anterior and posterior surfaces of the victim. First aid may be administered to treat the wounds on one surface, but other wounds on another surface may be more difficult to attend to. For example, a victim's wounds on the front and sides of his legs may be treated while the victim is laying on his back, but the wounds on the back of his legs may be inaccessible because of the need to maintain the victim face up, particularly during transport of the victim to a medical facility. Furthermore, wounds on the posterior surfaces of a victim's arms, legs, and head may be completely overlooked during transport of the victim and may only become apparent upon closer inspection when the victim is moved from a gurney or stretcher to an operating table. This problem is further exacerbated in cases where a victim is covered in blood and other debris as is sometimes the case in automobile accidents.

Based on the foregoing, it is the general object of the present invention to provide devices for controlling bleeding and methods of their use that overcome or improve upon the prior art.

### SUMMARY OF THE INVENTION

Disclosed herein are devices directed to the clotting of blood and the dressing of wounds. The devices generally comprise bandages that can be worn directly on wounds and that contain oxidized cellulose materials that can minimize or stop the flow of blood by absorbing at least portions of the liquid phases of the blood, thereby promoting clotting. When applied to a wound, the oxidized cellulose materials accelerate hemostasis by facilitating the formation of a coagulum plug over the wound. Although the devices are particularly suited for use in treating wounds resulting from traumatic injury, the present invention is not limited in this regard and the devices can be used in various surgical procedures in which it is desired to arrest the flow of blood.

According to one aspect, the present invention resides in an apparatus for promoting hemostasis that includes oxidized cellulose in the form of a compressible, shapeable mass that is formed into a sheet for placement on a bleed site. The oxidized cellulose is in strand form and may be woven or unwoven. When the apparatus is placed onto the bleed site and is contacted with blood flowing therefrom, a clotting effect is realized.

In a second aspect, the present invention resides in a sleeve for promoting hemostasis. The sleeve includes a tubular shell having at least one open end and oxidized cellulose material attached to an inner surface of the tubular shell. The open end of the tubular shell is dimensioned to receive a limb such that when treating a bleeding wound, donning the sleeve causes at least a portion of the oxidized cellulose material to be placed in contact with the bleeding wound. Upon contacting the bleeding wound with the oxidized cellulose material, a clotting effect is realized.

In a third aspect, the present invention resides in a bandage applicable to a bleeding wound. The bandage includes a substrate and oxidized cellulose material attached to a surface of the substrate. The oxidized cellulose material is arranged to contact the bleeding wound when the bandage is applied thereto. An adhesive may be applied to the substrate as needed to adhere the bandage to skin adjacent the wound, thereby facilitating the retention of the oxidized cellulose material in contact with tissue of the bleeding wound.

In any embodiment of the present invention, pharmaceutically-active compositions may be incorporated into the oxidized cellulose. Such pharmaceutically-active compositions include, but are not limited to, antibiotics, antifungal agents, antimicrobial agents, anti-inflammatory agents, anti-viral agents, analgesics, bone morphogenic proteins, bone-stimulating agents, compounds containing silver ions, corticosteroids, osteoblast stimulators, odontoblast stimulators, ascorbic acid, tranexamic acid, rutin, thrombin, botanical agents, and combinations of the foregoing.

One advantage of the present invention is that the devices can be readily removed from their packaging and either self-administered or administered by care-giving personnel in a single step. More specifically, because the devices incorporate hemostatic or blood clotting materials incorporated therein in pre-measured amounts, the need for applications of ointments, salves, creams, or other compositions followed by dressings of bandages and the application of pressure (to initiate clotting) is eliminated. In effect, the treatment process is streamlined, which may be of particular advantage in instances in which treatment is administered as first aid for traumatic injury (e.g., in the back of an ambulance or on a battlefield) in transit to a hospital or in anticipation of more sophisticated medical care.

The oxidized cellulose of the devices of the present invention is also advantageously biocompatible with living tissue. This biocompatibility allows the oxidized cellulose, which is a form of cotton, to be resorbed into the wound during the healing process. Resorption of the oxidized cellulose material into the wound, particularly when the material is in unwoven form, produces no detrimental effects on the living tissue.

Still another advantage of the present invention is that the proper dose of oxidized cellulose material can be readily applied to an open wound. Especially when the device is a sleeve or the like containing oxidized cellulose material, the device can be readily removed from sterilized packaging and used to treat the wounds directly at the points from which blood emanates to initiate clotting of the blood without inadvertently applying material outside the wound area. Guesswork, estimation, or calculation of the amounts of oxidized cellulose material for application to a bleeding wound is eliminated. Accordingly, little or no material is wasted.

### BRIEF DESCRIPTION OF THE DRAWINGS

FIG. 1 is a magnified photographic image taken by a scanning electron micrograph of oxidized cellulose.

FIG. 2 is a perspective view of oxidized cellulose in sheet form of the present invention.

FIG. 3 is a perspective view of oxidized cellulose attached to a substrate to form a pad of the present invention.

FIG. 4 is a perspective view of an adhesive bandage, of the present invention, incorporating oxidized cellulose.

FIG. 5 is a perspective view of a pad, of the present invention, securable using hook-and-loop material and incorporating oxidized cellulose.

FIG. 6 is a perspective view of a pad, of the present invention, securable using straps and incorporating oxidized cellulose.

FIG. 7 is a perspective view of a pad, of the present invention, securable using straps and incorporating oxidized cellulose.

FIG. 8 is a perspective view of a sleeve, of the present invention, having oxidized cellulose attached to an inner surface thereof.

FIG. 9 is a perspective view of a sleeve, of the present invention, having straps for securing the sleeve to a limb of a wearer.

### DETAILED DESCRIPTION OF THE PREFERRED EMBODIMENTS

Oxidized cellulose is a chemically oxidized form of a common cellulose fiber such as cotton and is also known as cellulosic acid, absorbable cellulose, or polyanhydroglucuronic acid. The degree of oxidation of the fiber is a function of the carboxylation content of the fibrous cellulose material. In particular, as the number of carboxyl groups on the cellulose structure is increased, the oxidation content correspondingly increases.

Oxidized cellulose may be manufactured by the action of nitrogen dioxide gas (NO₂) on cellulose fiber. Other methods of manufacturing oxidized cellulose include oxidation of cellulose fiber with aqueous oxidizing agents such as hypochlorite salts, although the use of such agents is less preferred than the use of nitrogen dioxide gas.

One method of generating nitrogen dioxide gas is by the catalytic reaction of manganese dioxide or manganese disulfide on concentrated nitric acid. Any amount of nitrogen dioxide can be generated by the metered addition of nitric acid to the manganese dioxide or manganese disulfide catalyst. In such a reaction, dinitrogen tetroxide (N₂O₄), which is a dimer of nitrogen oxide, is also formed in addition to the nitrogen dioxide. The formation of the dimer does not have an interfering effect on the oxidation of the cellulose.

In this method of nitrogen dioxide generation, unaltered cellulose fibers are introduced into a reaction vessel, and concentrated nitric acid is metered into a second enclosed vessel containing manganese dioxide powder. Nitrogen dioxide gas is evolved, which is piped to the reaction vessel containing the cellulose fibers. Once the nitrogen dioxide gas is piped to the reaction vessel containing the cellulose fibers, the reaction vessel is purged with an excess amount of nitrogen dioxide and left sealed for 36 hours. This may alternatively be done in a pressurized environment of nitrogen dioxide. The oxidized cellulose is then removed and washed in dilute sodium bicarbonate solution, followed by multiple agitated rinses with distilled water. Alternatively, the oxidized cellulose may be degassed using other suitable means. The resulting oxidized cellulose is thus sufficiently carboxylated to provide a desirable hemostatic effect on a bleeding wound. The resulting fibers can also be autoclaved before use.

Another method of generating nitrogen dioxide gas is by the reaction of formaldehyde with concentrated nitric acid. This reaction, however, is not catalytic. In particular, formaldehyde is consumed in the reaction and is thus depleted. The formaldehyde readily reacts with the nitric acid to generate the nitrogen dioxide and the dimer. Again, the nitrogen dioxide gas is piped to the reaction vessel containing the cellulose fibers, and the reaction vessel is purged with excess nitrogen dioxide and sealed. The oxidized cellulose is removed, washed, and rinsed.

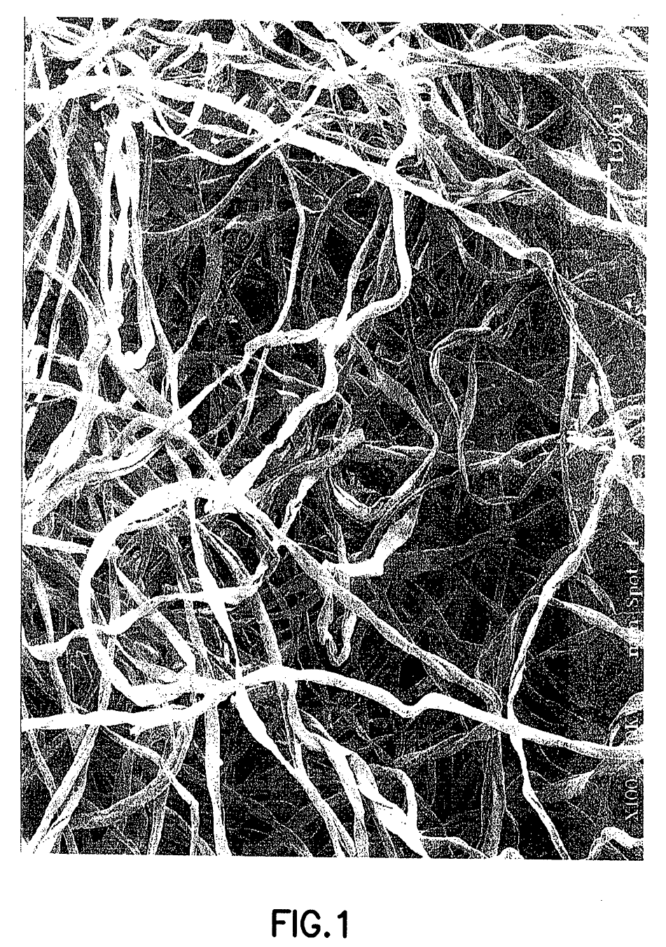

Referring to FIG. 1, the oxidized cellulose generated by either method is a mass of unwoven cellulose strands. The strands are loosely intermingled and easily compressed. The interstices between adjacent strands define areas in which the blood collects and the solids thereof agglomerate to facilitate the formation of clots.

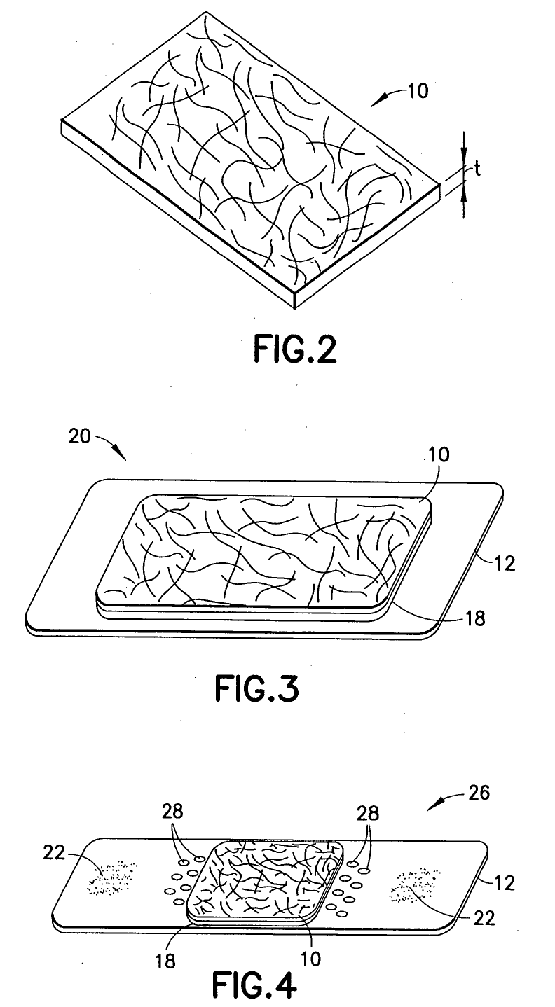

The compressibility of the unwoven cellulose strand mass allows the material to be formed into sheets that can be cut to any desired size and used as bandages. Referring now to FIG. 2, a sheet of compressed, unwoven, oxidized cellulose is shown generally at 10 and is hereinafter referred to as “sheet 10.” Sheet 10 is defined by a thickness t, which can vary depending upon the intended application. In particular, the thickness t can be thicker for applications in which heavy bleeding is encountered or thinner for applications in which an exudate from a wound is slight, such as from a burn or a mild abrasion of flesh. An increased thickness t provides enhanced compressibility to the sheet 10, which allows for increased absorbability of blood and other bodily fluids. Although shown as being rectangular, the sheet 10 can comprise any shape suitable or desired for application to a wound.

Referring now to FIG. 3, the sheet 10 may be attached to a substrate 12 to define a pad 20. Although the sheet 10 itself possesses some inherent absorptive qualities, an additional layer 18 of absorbent material may be disposed intermediate the substrate 12 and the sheet to absorb and accumulate excess blood and other bodily fluids that seep through the sheet when placed on a wound, thereby preventing or limiting wetting the substrate. The pad 20 may be approximately square as shown, or it may be elongated in a lengthwise dimension to approximate a rectangle. Optionally, the pad 20 may be an elongated strip and rolled during storage, thus being able to be unrolled and wound around a body part to contact a wound. By configuring the pad 20 as a rollable strip, the pad can be wound around the limbs, torso, or head of a wounded patient as necessary (e.g., intertwined among digits and the like).

The substrate 12 may be any suitable material conducive to being placed in proximity with a wound. Preferably, the substrate 12 is elastically deformable. This quality, in combination with the compressibility and elastic deformability of the oxidized cellulose sheet 10, allows the pad 20 to be wrapped tightly over portions of the wounded person's body so as to maintain pressure on the wound. Suitable materials for the substrate 12 include, but are not limited to, cloth (e.g., cotton, linen, wool, and the like) particularly in gauze form, metal, paper, polymeric material, synthetic or natural rubber, breathable synthetic materials (e.g., fluoropolymer-based material bonded to nylon or polyester fabric), combinations of the foregoing, and the like.

Referring now to FIG. 4, the substrate 12 may include an adhesive 22 disposed thereon to facilitate the attachment of the substrate to the skin adjacent the wound, thereby defining an adhesive bandage 26. The substrate 12 may include holes 28 formed or otherwise disposed therein to allow moisture (perspiration) to evaporate from the skin and to allow air to circulate to the wound when the bandage 26 is worn. The layer 18 of absorbent material may or may not be disposed intermediate the substrate 12 and the sheet 10. Once adhered to the skin of the user, the oxidized cellulose of the sheet 10 is held into direct contact with the wound, thereby initiating the clotting of blood.

Referring now to FIGS. 5-7, fasteners may be used to allow the substrate to be held on the skin adjacent the wound. In FIG. 5, one exemplary embodiment of a pad is depicted generally at 120. The pad 120 includes the sheet 10 of compressed, unwoven, oxidized cellulose attached to an elastically deformable substrate 112 having holes 128. Fasteners, such as hook-and-loop material, are used to secure the pad 120 to itself adjacent the wound. As is shown, the hook-and-loop material includes a hook material portion 125 attached proximate an edge of the substrate 112 and a loop material portion 127 attached proximate a second edge of the substrate. Although the substrate 112 is referred to as being elastically deformable, the present invention is not limited in this regard, and the substrate may be non-elastic.

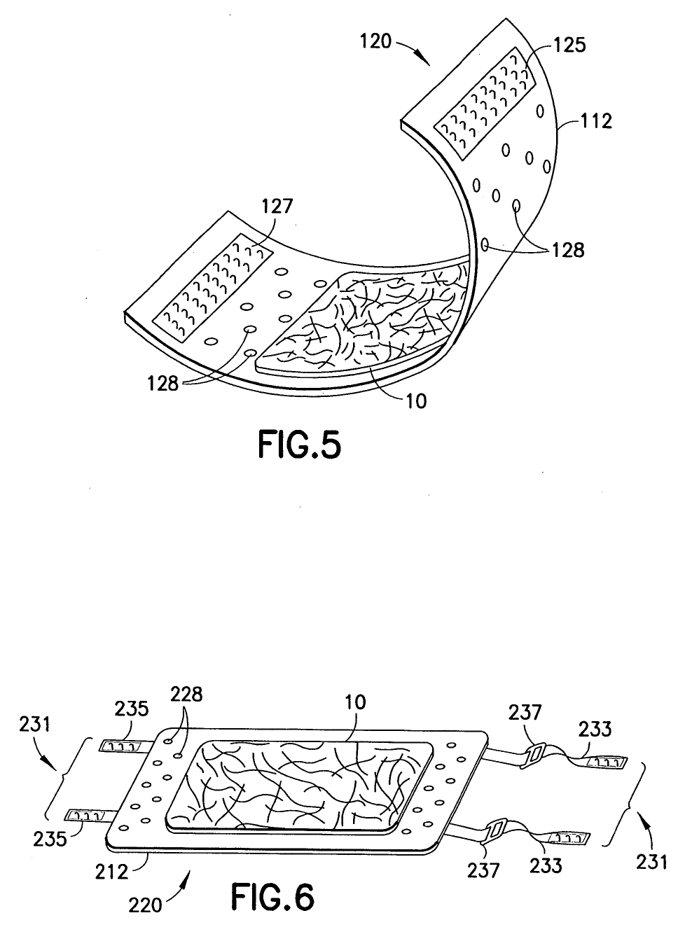

Referring to FIG. 6, another exemplary embodiment of a pad is depicted generally at 220. The pad 220 includes the sheet 10 of oxidized cellulose attached to an elastically deformable substrate 212 having holes 228. Fastening straps, depicted generally by reference numeral 231, are used to secure the pad 220 to itself adjacent the wound. The fastening straps 231, two of which are shown, each comprise a first member 233 and a second member 235, the first member being connectable to the second member via any suitable device such as hook-and-loop material or the like. The first members 233 are generally attached to the substrate 212 proximate an edge that is opposite the edge at which the second members 235 are attached. Preferably, at least the first member 233 is adjustable in length using a buckle 237. Although the substrate 212 is referred to as being elastically deformable, the present invention is not limited in this regard, and the substrate may be non-elastic.

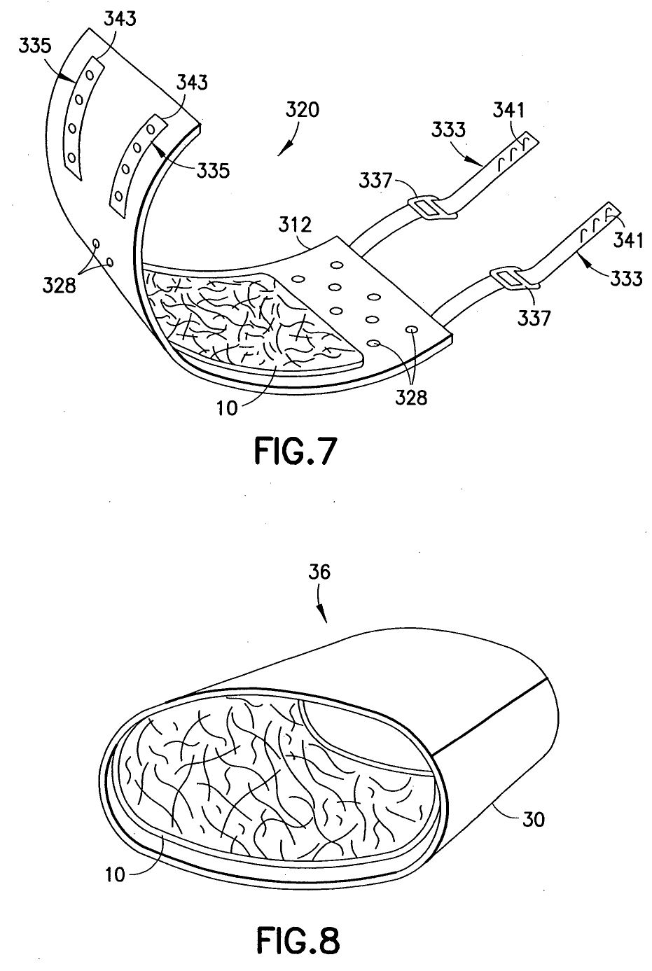

In FIG. 7, another exemplary embodiment of a pad is depicted generally at 320. The pad 320 includes the sheet 10 of oxidized cellulose attached to an elastically deformable substrate 312 having holes 328. Fastening straps 331 are used to secure the pad 320 to itself adjacent the wound. The fastening straps 333, two of which are shown, each comprise a first member 333 that is attachable to a second member 335 mounted to the substrate 312. Devices that may be used to facilitate the connection of the fastening straps 333 to the second members 335 include, but are not limited to, hooks 341 (on the fastening straps) and eyelets 343 (on the second members). Preferably, the fastening straps 333 are adjustable in length using a buckle 337. Although the substrate 312 is referred to as being elastically deformable, the present invention is not limited in this regard, and the substrate may be non-elastic.

Referring now to FIG. 8, the sheet 10 is positioned adjacent a tubular shell 30 to define a sleeve 36. The tubular shell 30 can be a nylon outer surface with an inner lining stitched thereto and filled with a compressible filler material. The sheet 10 is stitched, glued, or otherwise mounted to an inner surface of the tubular shell 30 (with or without the layer of absorbent material positioned therebetween). The tubular shell 30 is dimensioned such that it can be pulled over a limb, the torso, or even the head of a wounded person to bring the oxidized cellulose material of the sheet into contact with a wound.

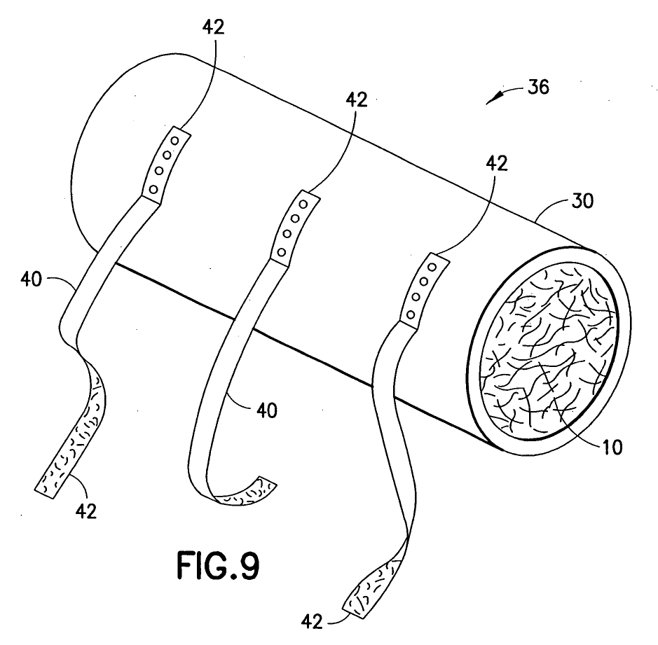

In some embodiments, the sleeve 36 may be securable to the arm, leg, or torso of a victim via one or more straps 40, as shown in FIG. 9. Portions of the straps 40 may be affixed to the outer surface of the tubular shell 30 at various points along the length of the sleeve 36 such that belt portions of the straps 40 may be wrapped around the sleeve and secured upon themselves. The straps 40 may be secured using hook-and-loop material 42, with one of the hook portion and the loop portion being disposed along the lengths of the belt portions and the other of the hook portion and the loop portion being disposed on the outer surface of the tubular shell 30. Alternately, the straps 40 may be nylon webbing or similar material, adjustable lengthwise using buckles, and securable to themselves using buttons, hooks, or quick-disconnect fasteners.

To utilize the sleeve 36 to dress a bleeding wound, the sleeve is removed from the packaging (if any) and held such that one end of the tubular shell 30 is opened. The sleeve 36 is then pulled over the wounded person's leg or arm such that the wound is covered. If the sleeve 36 has straps 40 attached to the outer surfaces of the tubular shell 30, the straps may be wrapped around the tubular shell and secured upon themselves. Once positioned on the victim, the oxidized cellulose in the sheet 10 contacts the tissue of the wound and/or the blood, and at least a portion of the liquid phase of the blood is adsorbed by the oxidized cellulose, thereby promoting clotting of the blood.

Other forms of the oxidized cellulose, such as those in woven form, are within the scope of the invention and suitable for use as sheet 10 in any of the above-described embodiments. After manufacture of any of the devices described above, the devices are preferably packed into containers that ensure the sterility of the devices until the containers are opened.

Various materials may be mixed with, associated with, disseminated throughout, embedded into, or incorporated into the oxidized cellulose to maintain an antiseptic environment or to provide functions that are supplemental to the hemostatic functions of the oxidized cellulose. Such materials can be used to stimulate cells, to promote adhesion to soft tissue (e.g., mucosa), or to promote adhesion to hard tissue (e.g., bone) to prevent dislodgement of the coagulum plug during the healing process.

One particular use may be for the acceleration of the healing process by effecting a timed release of medication into the tissue adjacent the wound. Accordingly, the material is dispensed into the wound simultaneously with the clotting function. Exemplary materials that can be used include, but are not limited to, pharmaceutically-active compositions such as antibiotics, antifungal agents, antimicrobial agents, anti-inflammatory agents, anti-viral agents, analgesics (e.g., cimetidine, chloropheniramine maleate, diphenhydramine hydrochloride, and promethazine hydrochloride), bone morphogenic proteins, bone-stimulating agents, compounds containing silver ions, corticosteroids, osteoblast stimulators, odontoblast stimulators, and the like. Other materials that can be incorporated to provide additional hemostatic functions include ascorbic acid, tranexamic acid, rutin, and thrombin. Botanical agents having desirable effects on the wound site may also be added.

After the cellulose is oxidized and either before or after it has been shaped into a device having a particular form (sheet 10 or the like), the drugs or other ancillary materials may be mixed with, associated with, disseminated throughout, embedded into, or incorporated into the cellulose using any suitable technique. Some methods may be as simple as shaking a dry powder into the fibrous matrix of the cellulose. Other methods may involve soaking the cellulose in aqueous solutions of the particular medication. Still other methods involve pressing the materials in slurry or particle form into the device. Liquid medications can also be absorbed into the fibers immediately prior to application of the device for subsequent delivery to a wound. Multiple ways exist for incorporation of these materials into the oxidized cellulose devices of the present invention, as will become evident during practice.

When placed on or in a wound, hemostasis can be established while the one or more pharmaceutically-active compositions are released into the wound. Upon removal of the device, any oxidized cellulose adhered to the coagulum plug can remain in place and be physiologically resorbed by the body.

Additionally, if the oxidized cellulose is configured to have a particular orientation in the wound (e.g., if the material is rolled or cut to fit into a laceration or puncture), the pharmaceutically-active composition may be located so as to effect a “site specific” delivery. For example, when a sheet of oxidized cellulose material is used to pack an open wound created by the removal or disruption of some amount of flesh, the pharmaceutically-active composition may be concentrated in the material around a peripheral edge thereof or in an outer layer, thus administering an application of the composition directly to the tissue of the wound without providing excess composition (e.g., in the center of the sheet) that is unlikely to have an effect on the healing process.

There are multiple clinical applications for the oxidized cellulose. It is especially indicated for first aid applications, namely for arresting the flow of blood from minor lacerations and abrasions and accelerating hemostasis. It is also suitable for providing an immediate cessation of blood flow resulting from traumatic injury, such as more severe lacerations and abrasions as well as puncture wounds. In both medical and dental surgical applications, it can be utilized in to fill voids that result from cyst removal, to deliver drugs after bone surgery, to deliver drugs that promote the healing of pulp after pulp exposures and other clinical cases in which hemostasis is desired with the added benefit of delivering drugs for the purpose of controlling infections and the acceleration of healing. It is also especially indicated for patients who have a tendency to profusely bleed such as hemophiliac patients or patients taking blood thinning medications. In veterinary practice, the devices can be especially useful in less-than-septic conditions, e.g., in barnyards, kennels, and the like. A myriad of other uses of this device will become apparent during routine use by medical, dental, and veterinary practitioners.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

1. An apparatus for promoting hemostasis, comprising: oxidized cellulose in the form of a compressible, shapeable mass that is formed into a sheet for placement on a bleed site.

2. The apparatus of claim 1, wherein said oxidized cellulose is in strand form and unwoven.

3. The apparatus of claim 1, wherein said oxidized cellulose is in strand form and woven.

4. The apparatus of claim 1, wherein said oxidized cellulose is carboxylated fibrous cellulose material.

5. The apparatus of claim 4, wherein said fibrous cellulose material is cotton.

6. The apparatus of claim 1, wherein said sheet is in strip form and rollable.

7. The apparatus of claim 1, further comprising a pharmaceutically-active composition incorporated into said oxidized cellulose, said pharmaceutically-active composition being selected from the group consisting of antibiotics, antifungal agents, antimicrobial agents, anti-inflammatory agents, anti-viral agents, analgesics, bone morphogenic proteins, bone-stimulating agents, compounds containing silver ions, corticosteroids, osteoblast stimulators, odontoblast stimulators, ascorbic acid, tranexamic acid, rutin, thrombin, botanical agents, and combinations of the foregoing.

8. The apparatus of claim 1, further comprising a substrate attached to said sheet of oxidized cellulose.

9. The apparatus of claim 8, wherein said substrate comprises an elastically deform able material.

10. The apparatus of claim 9, wherein said sheet is securable at a location adjacent said bleed site using a fastener selected from the group consisting of hook-and-loop material, buckles, hooks and eyelets, and combinations of the foregoing.

11. The apparatus of claim 8, wherein said substrate is selected from the group consisting of cloth, metal, paper, polymeric material, synthetic, rubber, natural rubber, breathable synthetic materials, and combinations of the foregoing.

12. The apparatus of claim 8, further comprising an adhesive disposed on said substrate.

13. The apparatus of claim 8, wherein said substrate is a tubular shell and wherein said sheet of oxidized cellulose is attached to an inner surface of said tubular shell.

14. The apparatus of claim 13, further comprising a strap having a first end attached to said tubular shell and a second end attachable back to said tubular shell.

15. A sleeve for promoting hemostasis, said sleeve comprising:
a tubular shell having an open end into which a limb can be inserted; and
oxidized cellulose material attached to an inner surface of said tubular shell;
wherein when treating a bleeding wound, donning said sleeve causes at least a portion of said oxidized cellulose material to be placed in contact with said bleeding wound.

16. The sleeve of claim 15, wherein said oxidized cellulose material is in strand form and unwoven.

17. The sleeve of claim 15, wherein said oxidized cellulose material is in strand form and woven.

18. The sleeve of claim 15, wherein said oxidized cellulose material is in the form of a sheet.

19. The sleeve of claim 15, further comprising a strap, said strap having a first end attached to said tubular shell and a second end attachable back to said tubular shell.

20. The sleeve of claim 19, wherein said second end of said strap is attachable back to said tubular shell using a device selected from the group consisting of hook-and-loop material, buckles, and quick-disconnect fasteners.

21. A bandage applicable to a bleeding wound, said bandage comprising:
a substrate; and
oxidized cellulose material attached to a surface of said substrate;
wherein said oxidized cellulose material is arranged to contact said bleeding wound when said bandage is applied to said bleeding wound.

22. The bandage of claim 21, further comprising an adhesive disposed on said surface of said substrate, said adhesive being capable of adhering said bandage to skin adjacent said bleeding wound.

23. The bandage of claim 21, further comprising an absorbent material disposed intermediate said substrate and said oxidized cellulose material.

24. The bandage of claim 21, wherein said oxidized cellulose material is in strand form and unwoven.

25. The bandage of claim 21, wherein said oxidized cellulose material is in strand form and woven.
